(19) United States
(12) Patent Application Publication
(10) Pub. No.: US 2014/0257130 A1
(43) Pub. Date: Sep. 11, 2014

(54) POWERED PULL WIRE DESIGN FOR ABLATION CATHETERS

(71) Applicant: BOSTON SCIENTIFIC SCIMED, INC., Maple Grove, MN (US)

(72) Inventors: HONG CAO, MAPLE GROVE, MN (US); KENNETH R. LARSON, GRAND RAPIDS, MN (US); HUISUN WANG, MAPLE GROVE, MN (US); JOHN C. BUMP, COON RAPIDS, MN (US)

(73) Assignee: BOSTON SCIENTIFIC SCIMED, INC., Maple Grove, MN (US)

(21) Appl. No.: 14/194,426

(22) Filed: Feb. 28, 2014

Related U.S. Application Data
(60) Provisional application No. 61/776,613, filed on Mar. 11, 2013.

(51) Int. Cl. A61B 18/14; A61B 5/00; A61B 5/01

(52) U.S. Cl. CPC A61B 18/1492; A61B 5/01; A61B 5/6852
USPC 600/549; 606/41; 606/33

(57) ABSTRACT

Medical devices such as pull wire catheters and methods for making and using medical devices are disclosed. A pull wire catheter may include an elongate tubular member having a proximal end region, a distal end region and a lumen extending between the proximal end region and the distal end region. A therapeutic element may be disposed at the distal end region. A pull wire may extend through the lumen to the distal end region and may be fixed to the distal end region such that relative movement between the pull wire and the tubular member proximal region alters the distal end region. The pull wire may have a core comprising a first material having a first conductivity and a cladding comprising a second material having a second conductivity.

### CROSS-REFERENCE TO RELATED APPLICATIONS

This application claims priority under 35 U.S.C. §119 to U.S. Provisional Application Ser. No. 61/776,613, filed Mar. 11, 2013, the entirety of which is incorporated herein by reference.

### FIELD

The present disclosure relates to medical devices and their methods of use. More specifically, the present disclosure relates to catheters having one or more elements at the distal end that are electrically connected proximally, and also having a pull wire for actuating a distal portion of the catheter. One example of such a device is a powered ablation catheter with a pull wire.

### BACKGROUND

A wide variety of intracorporeal medical devices have been developed for medical use, for example, intravascular use. Some of these devices include guidewires, catheters, and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### SUMMARY

Medical devices and methods for making and using medical devices are disclosed.

In general, the embodiments described herein use a powered pull wire, which is to say that one wire is configured to be suitable for use both as a pull wire and as a wire for electrical conduction. A pull wire may be made suitable for use as a wire for electrical conduction by providing a conductive cladding over the pull wire.

For example, one embodiment relates to a pull wire ablation catheter that includes a thermocouple sensor. The pull wire has, for example, a stainless steel core and a copper cladding. The cladding is electrically connected to an ablation element and to a thermocouple joint. Another wire extends proximally from the thermocouple joint.

Another embodiment relates to a pull wire ablation catheter that includes a thermistor. The pull wire has, for example, a stainless steel core and a copper or gold cladding. Two other wires extend proximally from the thermistor or the ablation element. One of the cladding and the two wires is electrically connected to the ablation element and the other two of the cladding and the two wires are electrically connected to the thermistor.

The above summary of some example embodiments is not intended to describe each disclosed embodiment or every implementation of the invention.

### BRIEF DESCRIPTION OF DRAWINGS

The disclosure may be more completely understood in consideration of the following detailed description of various embodiments in connection with the accompanying drawings, in which:

FIG. 1 is a schematic view illustrating a pull wire ablation catheter; and

FIGS. 2-4 are cross sectional views of the distal portions of pull wire ablation catheter embodiments.

The above summary of some embodiments is not intended to describe each disclosed embodiment or every implementation of the present disclosure. The Figures, and Detailed Description, which follow, more particularly exemplify these embodiments.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (i.e., having the same function or result). In many instances, the term “about” may be indicative as including numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

Although some suitable dimensions, ranges and/or values pertaining to various components, features and/or specifications are disclosed, one of skill in the art, incited by the present disclosure, would understand desired dimensions, ranges and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The detailed description and the drawings, which are not necessarily to scale, depict illustrative embodiments and are not intended to limit the scope of the invention. The illustrative embodiments depicted are intended only as exemplary. Selected features of any illustrative embodiment may be incorporated into an additional embodiment unless clearly stated to the contrary.

A pull wire or a control wire may be in deflectable catheters to change the shape of the distal end or to deflect the distal end of the catheter in situ by pulling on a proximal portion of a pull wire operatively connected to the distal end region of the catheter. Pull wires may be made from stainless steel and may have a diameter ranging from 0.007″ to 0.012″, depending on the required pull force. Deflectable ablation catheters may also contain multiple other wires such as power wires, sensing wires and thermal sensing wires. For a catheter having a diameter of 7 French or greater, fitting the required wires into the lumen of the catheter is not normally an issue. However, for catheters that are miniaturized for use in smaller spaces, the dimension of the catheter lumen may become a limiting design consideration.

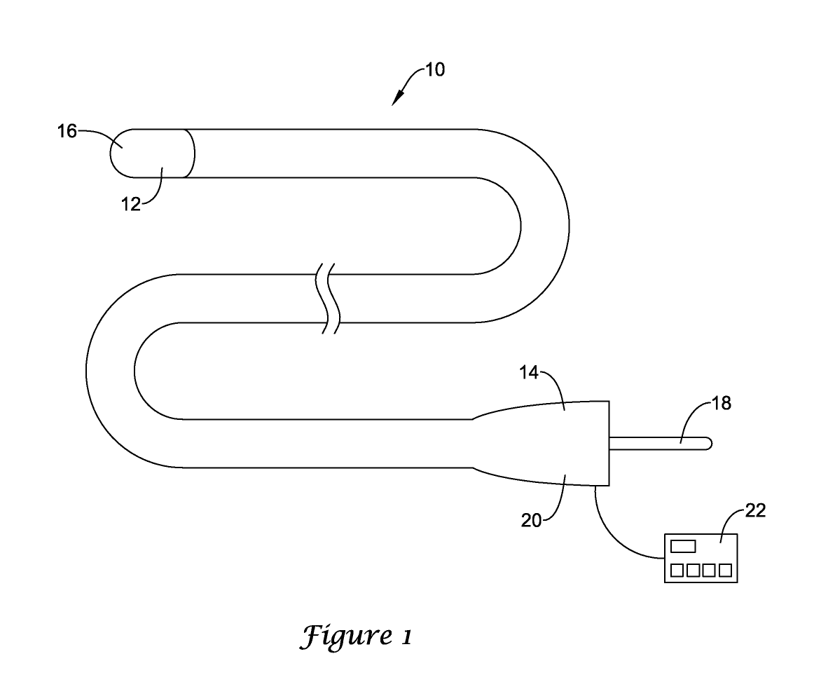

Turning now to FIG. 1, a therapeutic catheter 10 is depicted schematically. Catheter 10 has a distal end region 12, a proximal end region 14. An ablation element 16 is disposed at the distal end region 12. The ablation element 16 may be a radio frequency electrode, an ultrasonic transducer or other suitable ablation element. A pull wire 18 extends through the catheter 10 and may be fixed to the distal end region 12 and extend out from the proximal end region 14. Relative movement of the pull wire 18 and the proximal end region 14 may be used to deflect the distal end region, which may help with steering or with positioning the ablation element. A hub 20 may be disposed at the proximal end region 14 and may include connections to a power and control unit 22 or other typical connections.

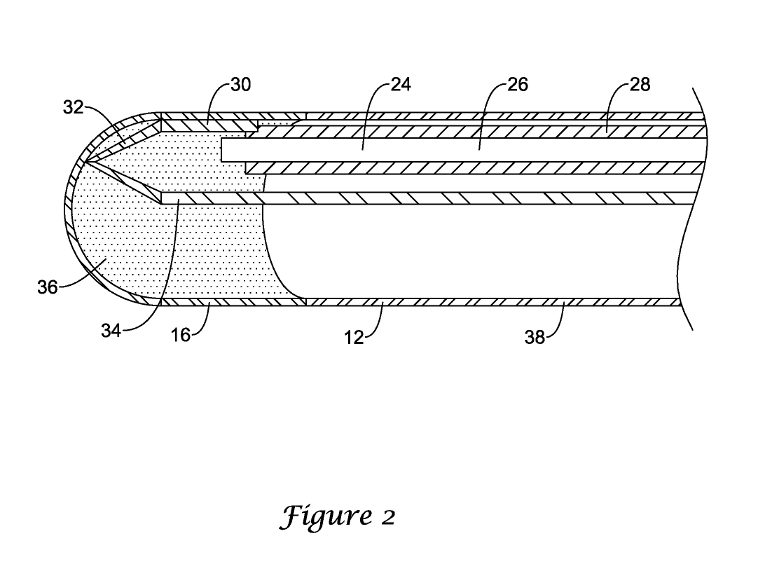

FIG. 2 is a cross-sectional view of the distal end region 12 of a therapeutic catheter 10 according to one embodiment. Within the catheter and extending to the ablation element 16 is a pull wire 24. Pull wire 24 may include a core 26 and a cladding 28. The cladding 28 may be electrically connected to the ablation element 16 by a conductor 30. Conductor 30 together with cladding 26 also acts as one of the wires of a thermocouple junction 32, of which wire 34 is the other wire of the thermocouple junction 32. An epoxy 36 or other suitable material may fill the distal end of the catheter and fix the pull wire 24 and thermocouple 32 in place. Epoxy 36 may be thermally conductive and electrically insulative. The thermocouple junction 32 may be placed against the ablation element 16 or embedded within the epoxy. The pull wire 24 may include an electrically insulating outer layer (not illustrated) extending proximally from the conductor 30. In some embodiments, the wire 34 may also include an electrically insulating outer layer (not illustrated) extending proximally from the thermocouple junction 32. The catheter includes a tube 38 extending proximally from the distal end region 12 to the proximal end region 14. In other embodiments, the ablation element may be a solid distal end of the catheter made from a solid metal such as platinum or a solid metal alloy such as a platinum iridium alloy, with machined holes for attaching wires or for providing irrigation.

The core 26 of the pull wire 24 should be suitable for use as a pull wire. Adequate tensile strength and anti-stretchability are two characteristics of a pull wire. Tensile strength refers to the maximum force in tension that the pull wire core can withstand. Anti-stretchability refers to the resistance to elongation of the material. A material with more resistance to stretching has a higher modulus of elasticity. Both properties, as discussed herein, are understood to be functions of the material(s), amount of materials and configuration. A suitable core 26 may be made from a solid stainless steel wire or from a cable made from strands of stainless steel.

The cladding 28 is used to conduct electricity. Suitable materials have a high conductivity. Copper and gold are suitable materials. Because the cladding is a layer on a core 26, the cladding can be quite thin and still be suitable for conducting electricity. In other words, the cross-sectional area of a pull wire core can be increased only slightly and provide equivalent electrical conduction to a separate wire much thicker than the cladding layer. For example, a pull wire having a 0.008″ stainless steel core with a cladding of 0.0005″ has lower resistance than a copper wire of diameter 0.004″. The cross-sectional area of the copper of the pull wire is greater than that of the solid copper wire. It can be appreciated that other configurations may be suitable for use as a combination pull wire and conductor. For example, the dimensions of the core and the cladding may be varied to meet desired engineering requirements. A 0.008″ stainless steel core with a 0.001″ copper cladding may be suitable in some applications. In other applications, the core may be made from the conductive material, and the cladding may be the less conductive layer having higher tensile strength. One example combination pull wire and conductor has a 0.003″ silver core with a 0.003″ stainless steel cladding. The cross-sectional dimensions of the core and the cladding determine their electrical resistance and yield strength.

Thus in one instance, FIG. 2 may depict the distal portion of a catheter where the pull wire 24 includes a stainless steel core 26 and a copper cladding 28. Conductor 30 may be copper as well. Wire 34 may be constantan. The junction of a copper wire and a constantan wire make a Type T thermocouple. When the combination pull wire and conductor is used as part of a thermocouple, it can be appreciated that one of the materials of the wire needs to be one of the two materials of the thermocouple.

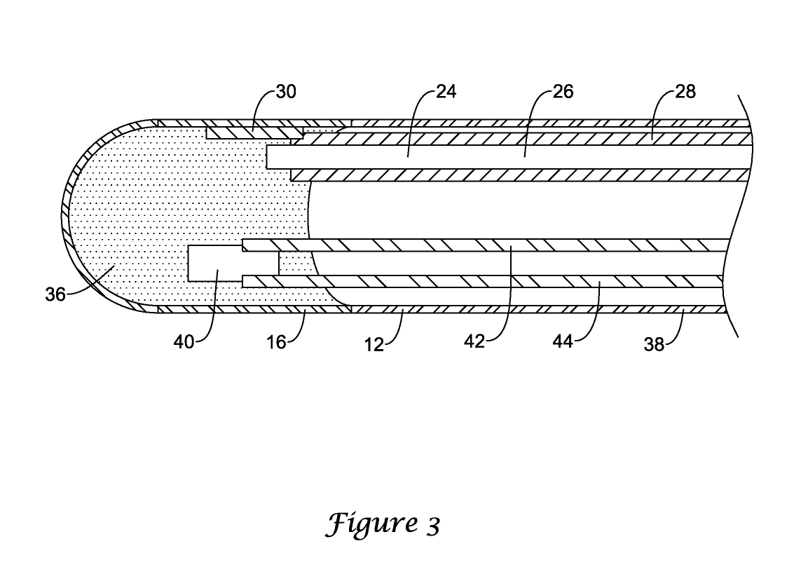

FIG. 3 illustrates another embodiment, similar to that of FIG. 2 except as discussed herein, where the pull wire 24 is electrically connected solely to the ablation element 16, and there are two wires 42, 44 extending from a sensing element 40. Sensing element 40 may be a thermistor, a thermocouple or other suitable sensor. The cladding 26 on the pull wire 24 may be copper, gold, or other suitable conductive material. Wires 42, 44 and pull wire 24 may include electrically insulating layers (not illustrated).

In alternative embodiments, the pull wire 24 may not be connected directly to the ablation element. The pull wire 24 may be attached to another structural element at or near the distal tip such as a pull ring. In such embodiments, an electrical conductor such as a short wire may need to extend distally of the pull wire to electrically connect pull wire to the ablation element or the pull wire may be mechanically connected to the pull ring or other structural element and extend distally past the pull ring to electrically connect to the ablation element.

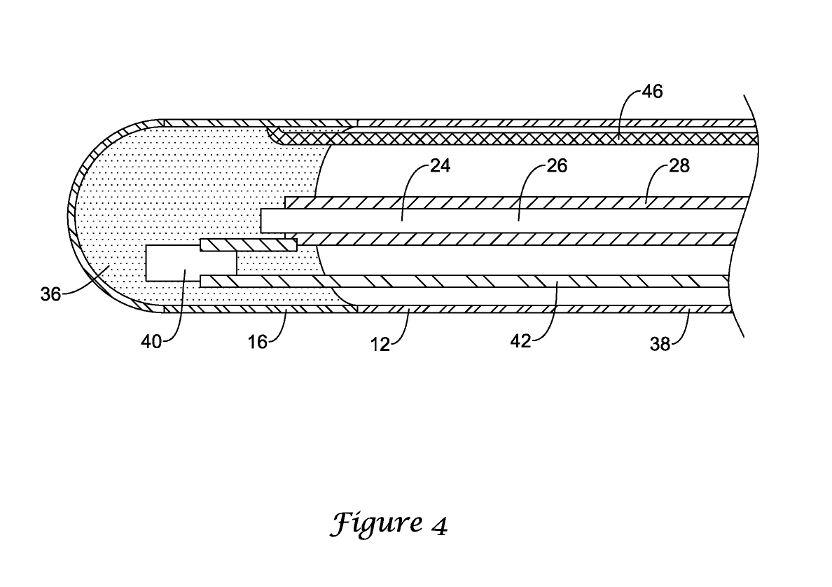

FIG. 4 illustrates another embodiment, similar to those of FIGS. 2 and 3 except as discussed herein, where a power wire 46 is electrically connected solely to the ablation element 16 and the pull wire 24 acts as one of the wires of the sensing element 40. Another wire 42 may also be connected to the sensing element.

At the proximal end of the catheter 10, the conductive elements such as cladding 26 and wires 34, 42 and 44 may be connected electrically to the power and control unit 22 through the hub or other suitable means. It will be appreciated that the cladding 26, because it is a part of pull wire 24, may move relative to the hub. A flexible wire that has enough play may therefore be placed between the pull wire and the hub to accommodate this movement. Other suitable methods of electrically connecting the pull wire to the power and control unit are also contemplated.

It will be appreciated that the use of a clad pull wire as described herein may readily be adapted to any medical device having a pull wire and a therapeutic element requiring electrical connectivity. It can also be appreciated that in devices that include more than one pull wire, each pull wire may be clad and used as an electrical wire as described herein. Further, there are catheters that include one or more stiffeners; it can be appreciated that these stiffeners may be clad and used as electrical elements. In other words, though the embodiments are described with respect to the combination of an actuatable pull wire and an electrical wire, embodiments are contemplated in which the “pull wire” is a fixed member.

In use, such a catheter 10 may be introduced percutaneously as is conventional, as for example, through a guide catheter. The pull wire may be actuated to deflect the distal end to aid in steering the catheter to the desired location within the body. When in place, the ablation element 16 is activated as desired. The catheter 10 may be repositioned and the procedure repeated.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A pull wire catheter, comprising:
an elongate tubular member having a proximal end region, a distal end region and a lumen extending between the proximal end region and the distal end region;
a therapeutic element disposed at the distal end region; and
a pull wire extending through the lumen to the distal end region and fixed to the distal end region such that relative movement between the pull wire and the tubular member proximal region alters the distal end region,
wherein the pull wire has a core comprising a first material having a first conductivity and a cladding comprising a second material having a second conductivity,
wherein the first material and the second material are selected such that the second conductivity is more than one order of magnitude higher than the first conductivity, and
wherein the cladding is electrically connected to the therapeutic element.

2. The pull wire catheter of claim 1, wherein the therapeutic element is a radio-frequency ablation element.

3. The pull wire catheter of claim 1, wherein the therapeutic element is an ultrasonic ablation element.

4. The pull wire catheter of claim 1, wherein the therapeutic element is a temperature sensor.

5. The pull wire catheter of claim 1, wherein the pull wire has a diameter of less than about 0.010″.

6. The pull wire catheter of claim 1, wherein the pull wire has a resistance of less than 5 ohms per millimeter.

7. The pull wire catheter of claim 1, wherein the pull wire has a stainless steel core.

8. The pull wire catheter of claim 1, wherein the pull wire has a copper cladding.

9. The pull wire catheter of claim 1, wherein the pull wire has a gold cladding.

10. The pull wire catheter of claim 1, further comprising:
a second therapeutic element at the distal end region; and
a conductor extending from the distal end region through the lumen, wherein the second therapeutic element is electrically connected to the conductor.

11. The pull wire catheter of claim 10, wherein the cladding is electrically connected to the second therapeutic element.

12. The pull wire catheter of claim 1, further comprising a power source electrically connected to the pull wire.

13. A pull wire catheter, comprising:
an elongate tubular member having a proximal end region, a distal end region and a lumen extending between the proximal end region and the distal end region;
an ablative element disposed at the distal end region;
a thermocouple at the distal end region;
an electrically conductive wire extending proximally from the distal end region; and
a pull wire extending through the lumen to the distal end region and fixed to the distal end region such that relative movement between the pull wire and the tubular member proximal region alters the distal end region,
wherein the pull wire has a core comprising a first material having a first conductivity and a cladding comprising a second material having a second conductivity,
wherein the first material and the second material are selected such that the second conductivity is more than one order of magnitude higher than the first conductivity, and
wherein the cladding is electrically connected to the ablative element and the thermocouple, and
wherein the pull wire is electrically connected to the thermocouple.

14. The pull wire catheter of claim 13, wherein the ablative element is a radio-frequency ablation electrode.

15. The pull wire catheter of claim 13, wherein the ablative element is an ultrasound transducer.

16. The pull wire catheter of claim 13, wherein the pull wire has a diameter of less than about 0.010″.

17. The pull wire catheter of claim 13, wherein the pull wire has a resistance of less than 5 ohms per millimeter.

18. The pull wire catheter of claim 13, wherein the pull wire has a stainless steel core.

19. The pull wire catheter of claim 13, wherein the cladding is copper and the pull wire is constantan.

20. A pull wire catheter, comprising:
an elongate tubular member having a proximal end region, a distal end region and a lumen extending between the proximal end region and the distal end region;
an ablative element at the distal end region;
a thermistor at the distal end region;
a first electrically conductive wire extending proximally from the distal end region;
a second electrically conductive wire extending proximally from the distal end region; and
a pull wire extending through the lumen to the distal end region and fixed to the distal end region such that relative movement between the pull wire and the tubular member proximal region alters the distal end region,
wherein the pull wire has a core comprising a first material having a first conductivity and a cladding comprising a second material having a second conductivity,
wherein the first material and the second material are selected such that the second conductivity is more than one order of magnitude higher than the first conductivity, and
wherein one of the cladding, the first electrically conductive wire and the second electrically conductive wire is electrically connected to the ablative element and the other two of the cladding, the first electrically conductive wire and the second electrically conductive wire are electrically connected to the thermistor.
